(19) United States
(12) Patent Application Publication
(10) Pub. No.: US 2017/0259026 A1
(43) Pub. Date: Sep. 14, 2017

(54) FILLING SYSTEM FOR AN ANESTHETIC DISPENSER

(71) Applicant: Dragerwerk AG & Co. KGaA, Lubeck (DE)

(72) Inventors: Franz MAIR, Lübeck (DE); Thomas LUTTER, Lübeck (DE); Michael HEIDSCHMIDT, Lübeck (DE)

(21) Appl. No.: 15/608,153

(22) Filed: May 30, 2017

Related U.S. Application Data
(63) Continuation of application No. 13/564,821, filed on Aug. 2, 2012, now abandoned.

(30) Foreign Application Priority Data
Feb. 2, 2012 (DE) 10 2012 001 965.3

(51) Int. Cl. A61M 16/18; B65B 3/04

(52) U.S. Cl. CPC A61M 16/183; B65B 3/04; A61M 2205/6045

(57) ABSTRACT

A filling system for an anesthetic dispenser has an improved anesthetic-specific code. The adapter neck of the cylinder adapter has polygonally arranged outer surfaces (7, 71) as anesthetic-specific code. A filling device for anesthetics at the anesthetic dispenser has a filler neck for receiving the adapter neck, and polygonally arranged inner surfaces (27, 271) are formed as an anesthetic-specific code at the filler neck, which are designed corresponding to the outer surfaces (7, 71). At least one inner surface (271) has a projection (272) projecting from the inner surface, and at least one outer surface (71) has a depression (72) designed corresponding to the projection (272).

### CROSS REFERENCE TO RELATED APPLICATIONS

This application is a continuation under 37 CFR 1.53(b) of pending prior application Ser. No. 13/564,821 filed Aug. 2, 2012 and claims the benefit of priority under 35 U.S.C. §119 of German Patent Application DE 10 2012 001 965.3 filed Feb. 2, 2012. The entire content of each application is hereby incorporated by reference in its entirety.

### FIELD OF THE INVENTION

The present invention pertains to a filling system for an anesthetic dispenser.

### BACKGROUND OF THE INVENTION

A filling system of this type is known from DE 10 2004 061 392 B3 (and corresponding U.S. Pat. No. 7,168,467 B2). A cylinder adapter, which is designed for connection to a storage tank for liquid anesthetic, has an anesthetic-specific code on the adapter neck in the form of an outer polygon. An inner polygon, which has a shape corresponding to that of the outer polygon, is provided on the filling device at the anesthetic evaporator.

A cylinder adapter, which establishes the connection between the storage tank for liquid anesthetic and the filling device at the anesthetic evaporator, is necessary in the filling device known from U.S. Pat. No. 6,585,016 B1. A threaded sleeve, which has two grooves for receiving an index collar arranged on the neck of the storage tank, is provided at the cylinder-side end of the cylinder adapter. The index collar has an anesthetic-specific design, so that only the cylinder adapter fitting the storage tank can be screwed onto the thread of the cylinder.

An anesthetic-specific code in the form of two projections, which are arranged angularly offset in relation to one another and which mesh with corresponding recesses on the filling neck of the filling device, are likewise provided on the adapter neck of the cylinder adapter.

The outlet pipe of the cylinder adapter is closed by means of a spring-loaded adapter valve, so that no anesthetic vapor can escape into the environment. A corresponding filling valve is provided at the filling device. A stationary rod, which has such a length that the adapter valve can be opened when the adapter neck is inserted into the filler neck of the filling device, is located in the middle of the filler neck of the filling device. The interaction between the cylinder adapter and the filling device is designed such that the filling valve opens first and the adapter valve thereafter. Liquid anesthetic can then flow from the storage tank into the tank of the anesthetic evaporator.

### SUMMARY OF THE INVENTION

A basic object of the present invention is to provide an improved anesthetic-specific code for a filling system of the type mentioned and for a cylinder adapter.

According to the invention, an anesthetic dispenser filling system is provided comprising a cylinder adapter with an adapter neck. The cylinder adapter is provided at a storage tank for liquid anesthetic. Polygonally arranged outer surfaces are provided at the adapter neck as an anesthetic-specific code at the cylinder adapter. The system also comprises an anesthetic dispenser. A filling device for anesthetic with a filler neck for receiving the adapter neck is provided at the anesthetic dispenser. Polygonally arranged inner surfaces, as an anesthetic-specific code at the filler neck, are provided that are designed corresponding to the polygonally arranged outer surfaces. At least one of the polygonally arranged inner surfaces has a projection projecting from the inner surface and at least one of the polygonally arranged outer surfaces has a depression designed corresponding to the projection.

The polygonally arranged inner surfaces and outer surfaces may provide a generally polygon shape designed corresponding to as a regular n-sided polygon. The polygonally arranged inner surfaces and outer surfaces may provide a generally polygon shape that has sides corresponding to one of a hexagon, a pentagon, a heptagon or an octagon.

The projection of the at least one inner surface may comprise a pin-shaped component and the depression at the at least one outer surface may comprise a groove corresponding to the pin-shaped component. The projection of the at least one inner surface may be a convex contour and the depression of the at least one outer surface may be an inwardly directed contour designed corresponding to the convex contour.

All of the inner surfaces may have a convex contour and all of the outer surfaces have an inwardly directed contour designed corresponding to the convex contour. The inwardly directed contour may be designed as a curvature in a radial direction of the polygonally arranged outer surfaces and the polygonally arranged inner surfaces wherein no curvature is present at right angles thereto.

According to another aspect of the invention, a cylinder adapter is provided for filling an anesthetic dispenser. The cylinder adapter comprises an adapter neck and polygonally arranged outer surfaces at the adapter neck as an anesthetic-specific code of the cylinder adapter. At least one of the outer surfaces has an inwardly directed contour.

All of the outer surfaces may have an inwardly directed contour. The contour may comprise a curvature in a radial direction of the polygonally arranged outer surfaces wherein there is no curvature present at right angles thereto.

The anesthetic-specific code is prepared in such a manner that polygonally arranged outer surfaces on the adapter neck of the cylinder adapter are present and the filling device has polygonally shaped inner surfaces designed correspondingly thereto. At least one of the inner surfaces is provided with a projection in the form of a lug or a convex contour and at least one of the outer surfaces has a depression adapted to the projection. This depression is designed such that the cylinder adapter can be inserted into the filling device when the at least one inner surface is in contact with the at least one outer surface. It is within the scope of the present invention that each of the inner surfaces has a projection and each of the outer surfaces has a depression or contraction designed correspondingly thereto. No special preferential position needs to be maintained for this case for inserting the cylinder adapter into the filling device, but the insertion position can be found by slightly rotating the cylinder adapter. The convex contour is especially advantageously designed as a convex arch.

The polygonal arrangement of the outer surfaces and inner surfaces is preferably embodied in the form of a regular n-sided polygon. A 5-sided polygon is thus used for sevoflurane, a 6-sided polygon for isoflurane, and an 8-sided polygon for halothane.

The convex contour on the inner surface and outer surface is designed such that it is a curvature in the radial direction of the polygon and no curvature is present at right angles thereto.

The scope of protection expressly covers cylinder adapters that have the code according to the present invention. The cylinder adapters can now be screwed onto the storage tank or especially preferably they can be firmly connected to the storage tank for liquid anesthetic or the cylinder adapter is fastened to the storage tank by means of a flange connection.

An exemplary embodiment of the present invention is shown in the figure and will be explained in more detail below. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

FIG. 1 is a perspective view of a cylinder adapter according to the invention and corresponding to the embodiments of FIGS. 5 and 6 wherein the view shows polygonally arranged outer surfaces, showing a side with the flat outer surfaces corresponding to the state of the art, wherein the at least one outer surface with a depression is at a side opposite the side being viewed;

FIG. 2 is a longitudinal sectional view of the cylinder adapter according to FIG. 1 and corresponding to the embodiments of FIGS. 5 and 6 wherein the view shows polygonally arranged outer surfaces, showing a side with the flat outer surfaces corresponding to the state of the art, wherein the at least one outer surface with the depression is at a side opposite the side being viewed;

FIG. 3 is a perspective view of a filling device according to the invention and corresponding to the embodiments of FIGS. 5 and 6 wherein the view shows polygonally arranged inner surfaces, showing a side with the flat inner surfaces corresponding to the state of the art, wherein the at least one inner surface with a projection is at a side opposite the side being viewed;

FIG. 4 is a longitudinal section of the filling device according to FIG. 3 and corresponding to the embodiments of FIGS. 5 and 6 wherein the view shows polygonally arranged inner surfaces, showing a side with the flat inner surfaces corresponding to the state of the art, wherein the at least one inner surface with the projection is at a side opposite the side being viewed;

FIG. 5 is a schematic view showing a first code with polygonally arranged surfaces including the at least one inner surface with the projection and the at least one outer surface with the depression;

FIG. 6 is a schematic view showing a second code with polygonally arranged surfaces including the at least one inner surface with the projection and the at least one outer surface with the depression;

FIG. 7 is a schematic view showing a third code with polygonally arranged surfaces including the all of the inner surfaces with projections and all of the outer surfaces with depressions;

### DESCRIPTION OF THE PREFERRED EMBODIMENTS

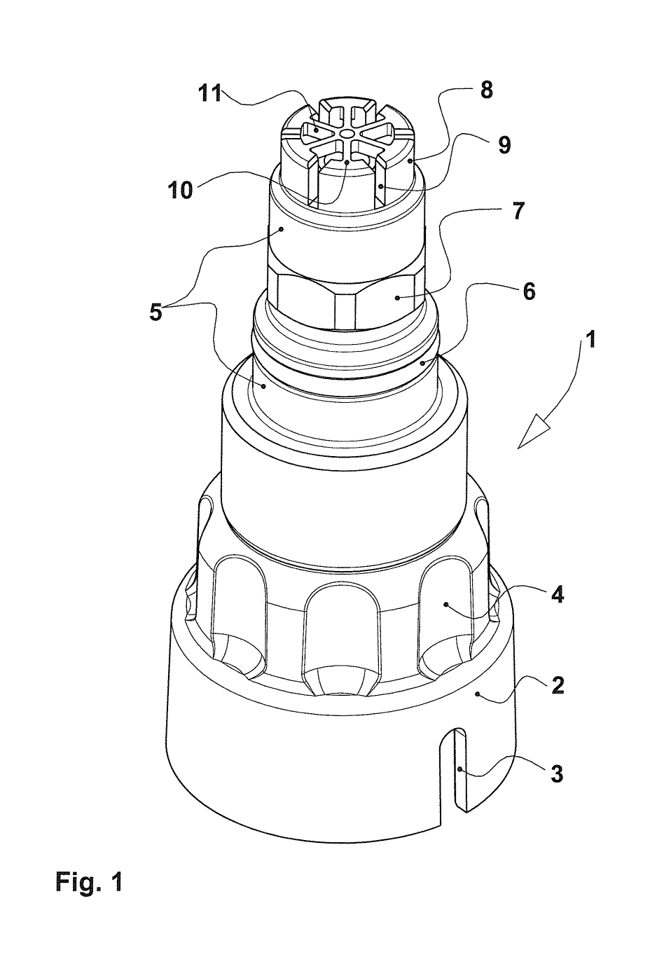

Referring to the drawings in particular, FIG. 1 shows a perspective view of a cylinder adapter 1, which has a threaded sleeve 2 on the underside for screwing onto a storage tank for liquid anesthetic, not shown specifically in FIG. 1. On its cylinder collar, the storage tank has an anesthetic-specific index collar, which is inserted into mounting grooves 3 of the threaded sleeve 2. Thus, only one cylinder adapter 1 belonging to the anesthetic can be screwed onto the storage tank. The screwing motion is facilitated here by recessed grips 4 above the threaded sleeve 2. An adapter neck 5 with an O-ring 6 and polygonally arranged outer surfaces 7 for the anesthetic-specific code as well as an outlet pipe 8 with radially extending slots 9 are located at the upper end of the cylinder adapter 1. The upper side 10 of the pipe is designed as a star-shaped body 11. The code comprises polygonally arranged surfaces 7/71 including the at least one outer surface 71 with the depression wherein only the flat outer surfaces 7, corresponding to the state of the art are viewable in FIG. 1.

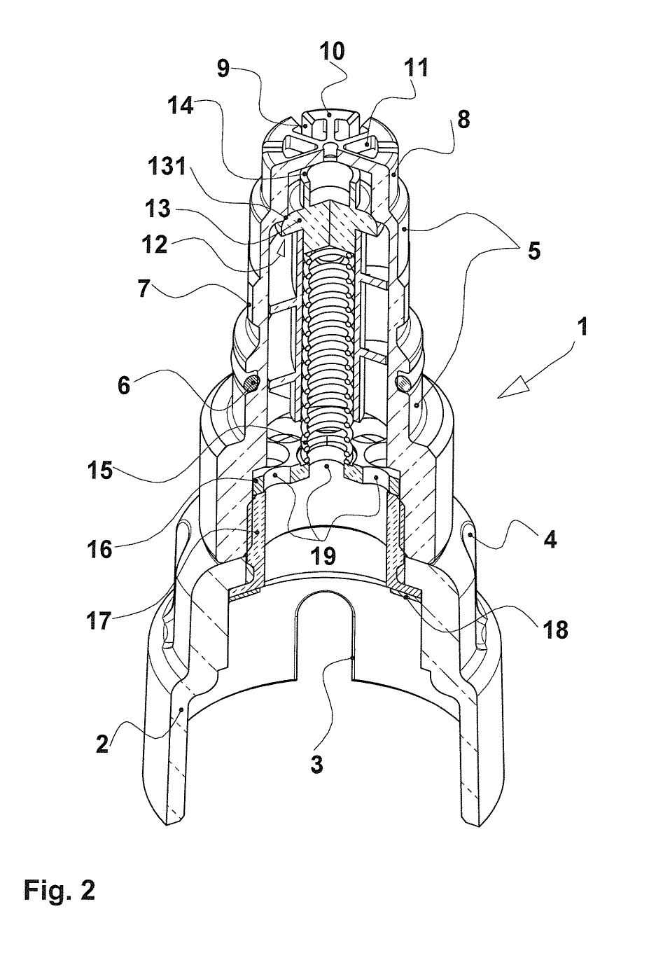

FIG. 2 shows the cylinder adapter 1 according to FIG. 1 in a longitudinal section. Identical components are designated by the same reference numbers as in FIG. 1. A shut-off valve 12 with a valve piston 13, with a ring body 14 on the upper side of the valve piston 13 and with a valve spring 15, which is prestressed by means of a support plate 16 and pushes the valve piston 13 against a sealing surface 131, is located within the cylinder adapter 1. Support plate 16 is held within the cylinder adapter 1 by a support cage 17. A sealing ring 18, which lies on the cylinder neck of the storage tank, not shown in the figures, is fastened to the underside of the support cage 17. Support plate 16 contains holes 19, via which the gas and liquid exchange takes place. The surfaces 7/71 generally provide a polygonal shape is of an anesthetic-specific design. Thus, a 5-sided generally polygonal shape is used for sevoflurane, a 6-sided generally polygonal shape for isoflurane, and an 8-sided generally polygonal shape for halothane.

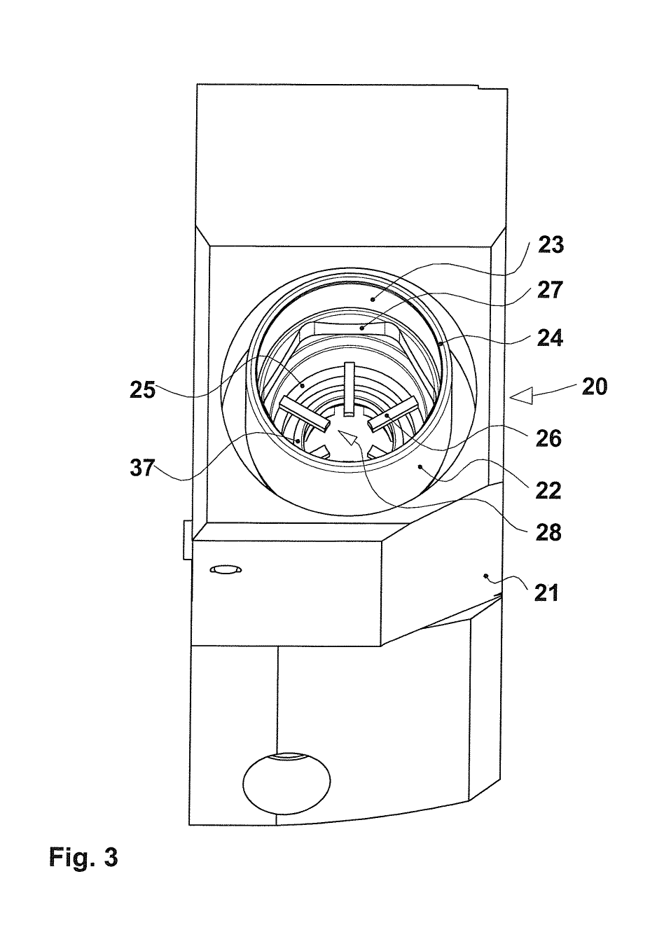

FIG. 3 shows a perspective view of a filling device 20 for anesthetic at an anesthetic dispenser 21. The filling device 20 has a filler neck 22 with a cylindrical sealing surface 23, an inner lead-in bevel 24 at the upper part of the filler neck 22, a spoked wheel 25 with radially inwardly pointing rods 26, polygonally arranged inner surfaces 27 for the anesthetic-specific code and a filling valve 28. FIG. 3 shows the code with polygonally arranged surfaces including the at least one inner surface with the projection wherein only the flat inner surfaces 27 corresponding to the state of the art are viewable in FIG. 3.

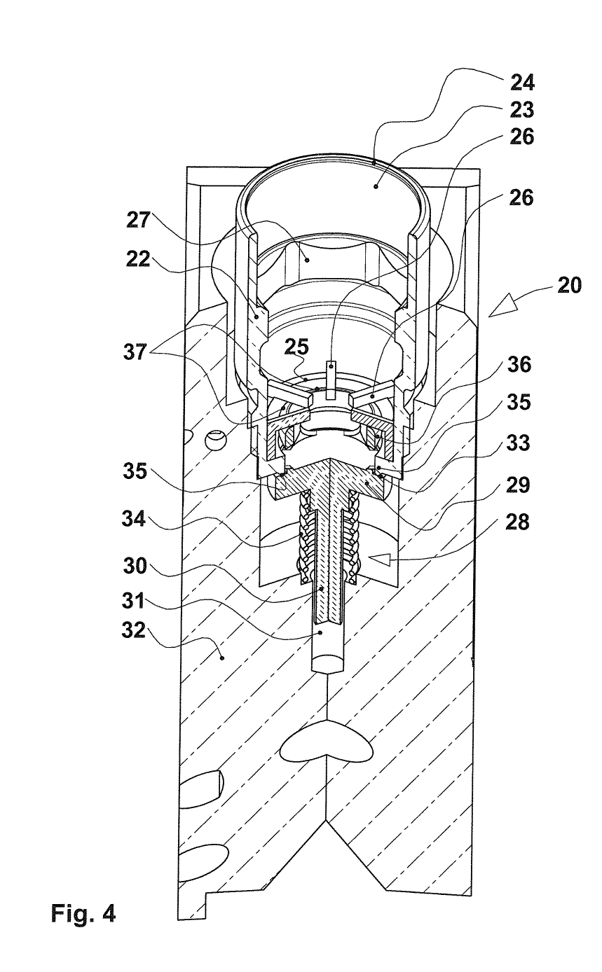

FIG. 4 shows a longitudinal section of the filling device 20. Identical components are designated by the same reference numbers as in FIG. 3. Filling valve 28 comprises a valve plate 29 at a valve guide rod 30, which is received in a hole 31 of the dispenser housing 32 such that it can perform strokes. The upper side 33 of the valve plate 29 is pressed by means of a valve spring 34 against a sealing crater 35 of the filling valve 28. A guide ring 36 is connected to the valve plate 29 and is displaceable together with valve plate 29.

The filling system operates as follows:

When the cylinder adapter 1 is inserted into the filling device 20, the O-ring 6 first slides over the lead-in bevel 24 and is in contact with the sealing surface 23 of filler neck 22. The outer surfaces 7 come to mesh in the further course with the inner surfaces 27 and the cylinder adapter 1 is centered as a result in relation to the filling device 20. The rods 26 are then located in the slots 9 of the outlet pipe 8 and the outlet pipe 8 enters the area of guide ring 36.

By a pressure being applied to the cylinder adapter 1, the upper side 10 of the pipe of the cylinder adapter is in contact with the upper side 37 of the guide ring 36 and the filling valve 28 opens. The adapter valve 12 is still closed. If the pressure on the cylinder adapter 1 is increased further, valve plate 29 is displaced against the force of the valve spring 34 further downward and the rods 26 touch the ring body 14 of the valve body 13, as a result of which the adapter valve 12 opens. Anesthetic is now flowing via the holes 19 of the support plate 16 into the tank of the anesthetic evaporator 21 and, as in communicating vessels, gas returns from the tank into the storage tank via the holes 19.

When ending the filling operation, the adapter valve 12 closes at first, so that possible residual quantities of anesthetic within the filler neck 22 can flow off into the tank of the anesthetic evaporator 21. The filling valve 28 is then closed and the cylinder adapter 1 can be removed from the filler neck 22.

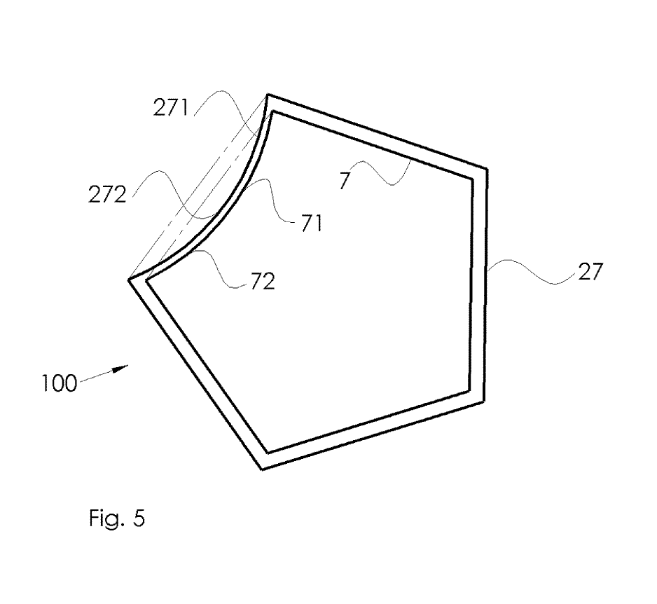

FIG. 5 schematically shows a first embodiment 100 of the code according to the present invention, and only the cooperation of the polygonally arranged outer surfaces 7 on the cylinder adapter 1 and the polygonally shaped inner surfaces 27 on the filling device 20 is shown. A first outer surface 71, being a partial surface of the outer surfaces 7, has an inwardly directed arch 72, and a first inner surface 271 designed correspondingly thereto has a convex arch 272. When the first outer surface 71 is aligned with the first inner surface 271, cylinder adapter 1 can be pushed into the filling device 20.

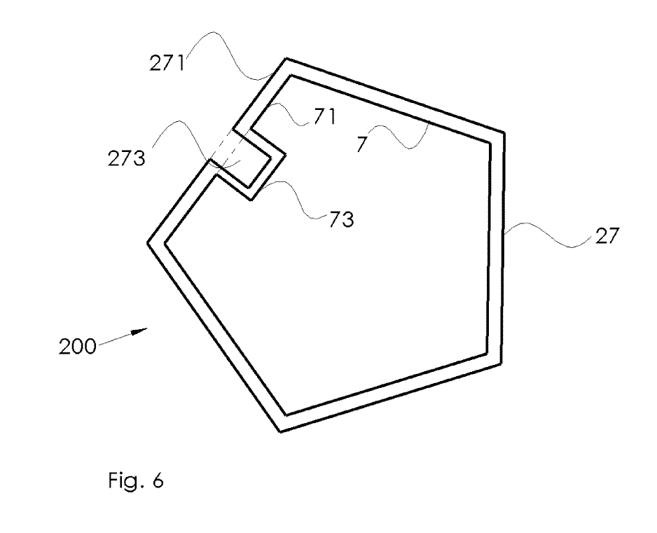

Unlike in the first embodiment according to FIG. 5, a pin-shaped component 273 is present at the first inner surface 271 in a second embodiment 200 shown in FIG. 6, and the first outer surface 71 has a groove 73 designed correspondingly thereto.

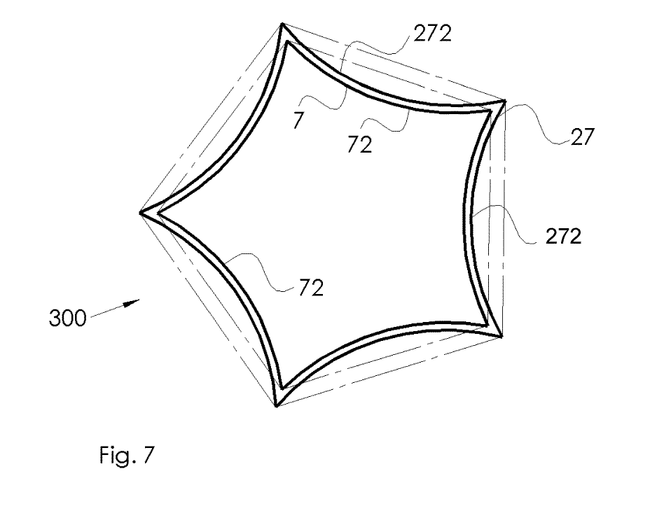

In a third embodiment 300 according to FIG. 7, all inner surfaces 27 have a convex arch 272 and all outer surfaces 7 are provided with the inwardly directed arch 72. Unlike in the first embodiment 100 with an arched partial surface, no special preferential position is needed in the third embodiment 300 for inserting the cylinder adapter 1 into the filling device 20.

While specific embodiments of the invention have been shown and described in detail to illustrate the application of the principles of the invention, it will be understood that the invention may be embodied otherwise without departing from such principles.

### APPENDIX

### List of Reference Numbers

1 Cylinder adapter
2 Threaded sleeve
3 Mounting grooves
4 Recessed grip
5 Adapter neck
6 O-ring
7 Outer surface
8 Outlet pipe
9 Slots
10 Upper side of pipe
11 Star-shaped body
12 Shut-off valve
13 Valve piston
14 Ring body
15 Valve spring
16 Support plate
17 Support cage
18 Sealing ring
19 Hole
20 Filling device
21 Anesthetic dispenser
22 Filler neck
23 Sealing surface
24 Lead-in bevel
25 Spoked wheel
26 Rods
27 Inner surface
28 Filling valve
29 Valve plate
30 Valve guide rod
31 Hole
32 Dispenser housing
33 Upper side
34 Valve spring
35 Sealing seat (crater)
36 Guide ring
71 First outer surface
72 Arch
73 Groove
100 First embodiment
131 Sealing surface
200 Second embodiment
271 First inner surface
272 Convex arch
273 Pin-shaped component
300 Third embodiment

What is claimed is:

1. An anesthetic dispenser filling system comprising:
a cylinder adapter with an adapter neck, said cylinder adapter being provided at a storage tank for liquid anesthetic;
polygonally arranged outer surfaces at said adapter neck as an anesthetic-specific code at the cylinder adapter;
an anesthetic dispenser;
a filling device for anesthetic with a filler neck for receiving said adapter neck, said filling device being at said anesthetic dispenser; and
polygonally arranged inner surfaces, as an anesthetic-specific code at said filler neck, said polygonally arranged inner surfaces being designed corresponding to said polygonally arranged outer surfaces, wherein at least one of said polygonally arranged inner surfaces has a projection projecting from the inner surface and at least one of said polygonally arranged outer surfaces has a depression designed corresponding to said projection.

2. A filling system in accordance with claim 1, wherein the polygonally arranged inner surfaces and outer surfaces provide a generally polygon shape designed corresponding to as a regular n-sided polygon.

3. A filling system in accordance with claim 1, wherein said polygonally arranged inner surfaces and outer surfaces provide a generally polygon shape with sides corresponding to one of a hexagon, a pentagon, a heptagon or an octagon.

4. A filling system in accordance with claim 1, wherein said projection of said at least one inner surface comprises a pin-shaped component and said depression at said at least one outer surface comprises a groove corresponding to said pin-shaped component.

5. A filling system in accordance with claim 1, wherein said projection of said at least one inner surface is a convex contour and said depression of said at least one outer surface is an inwardly directed contour designed corresponding to said convex contour.

6. A filling system in accordance with claim 5, wherein all of said inner surfaces have a convex contour and all of said outer surfaces have an inwardly directed contour designed corresponding to said convex contour.

7. A filling system in accordance with claim 5, wherein said convex contour and said inwardly directed contour are designed as a curvature in a radial direction of said polygonally arranged outer surfaces and said polygonally arranged inner surfaces and that no curvature is present at right angles thereto.

8. A cylinder adapter for filling an anesthetic dispenser, cylinder adapter comprising
an adapter neck; and
polygonally arranged outer surfaces at the adapter neck as an anesthetic-specific code of the cylinder adapter, wherein at least one of said outer surfaces has an inwardly directed contour.

9. A cylinder adapter in accordance with claim 7, wherein all of said outer surfaces have an inwardly directed contour.

10. A cylinder adapter in accordance with claim 8, wherein said contour comprises a curvature in a radial direction of said polygonally arranged outer surfaces and that no curvature is present at right angles thereto.
